(19) United States
(12) Patent Application Publication
(10) Pub. No.: US 2009/0155392 A1
(43) Pub. Date: Jun. 18, 2009

(54) Methods and Systems for Sublingual Guarana Administration

(76) Inventors: Bret David Nelson, Draper, UT (US); Richard Elijah Allred, Highland, UT (US)

Correspondence Address: KIRTON AND MCCONKIE, 60 EAST SOUTH TEMPLE,, SUITE 1800, SALT LAKE CITY, UT, 84111, US

(21) Appl. No.: 12/336,331

(22) Filed: Dec. 16, 2008

Related U.S. Application Data
(60) Provisional application No. 61/014,326, filed on Dec. 17, 2007.

(51) Int. Cl. A61K 36/77; A61P 3/00; A61P 25/00

(52) U.S. Cl. 424/725

(57) ABSTRACT

Embodiments of the present invention relate generally to methods and systems for the sublingual and buccal administration of herbal supplements, and more particularly, to the sublingual and buccal administration of Guaraná, which allows for considerably reducing the therapeutic dose, with the additional advantage of increasing the quickness of the beneficial effects.

### RELATED PATENT APPLICATIONS

This application claims priority to U.S. Provisional Patent Application No. 61/014,326, filed Dec. 17, 2007, the contents of which are being incorporated herein in their entirety.

### FIELD OF THE INVENTION

The present invention relates generally to methods and systems for the sublingual and buccal administration of herbal supplements, and more particularly, to the sublingual and buccal administration of Guaraná, which allows for an increase in the quickness of the beneficial effects as well as a reduction in the therapeutic dose required.

### BACKGROUND OF THE INVENTION

Guaraná (pronounced wah-rah-NAH) is a berry that grows in Venezuela and the northern parts of Brazil. The name ‘Guaraná’ comes from the Guarani tribe that lives in Brazil. Guaraná plays a very important role in their culture, as this herb is believed to be a cure for a variety of ailments and a way to regain strength. Guaraná's biological name, Paullinia Cupana, was taken from the German medical botanist C. F. Paullini, who discovered the tribe and the plant in the 18th century. The taste of guaraná is distinctive and unique, which is one reason for its recent success as a soft drink in Brazil and other countries.

Some of the many documented uses for guaraná include as a weight loss aid (suppresses appetite and increases fat-burning), athletic enhancement, concentration, endurance, exhaustion, fatigue, headaches and migraines, mental depression or irritation, water retention, to prevent overheating and heat stroke and as an aphrodisiac.

Other properties documented by research include as an: analgesic (pain-reliever), antibacterial, anticoagulant (blood thinner), antioxidant, hyperglycemic, memory enhancer, nervine (balances/calms nerves), neurasthenic (reduces nerve pain), platelet aggregation inhibitor (to prevent clogged arteries), stimulant, and vasodilator.

The main ingredient of guaraná is guaranine, which is chemically identical to caffeine. This may be one of the reasons for the energy boost people get after taking guaraná. The chemical composition of guaraná seeds include: Vegetable fiber: 49.125%, Reddish resin: 8.800%, Starch: 8.350%, Water: 7.650%, Pectin, malic acid, mucilage, dextrin, salts: 7.470%, Guaraná-tannic acid: 5.902%, Caffeine: 5.388%, Yellowish steady oil: 2.950%, Pyro-guaraná acid: 2.750%, Reddish colorant: 1.520%, Amorphous substances: 0.606%, and Saponin: 0.060%.

Since its discovery, the many benefits of guaraná have been passed on to explorers and settlers. European researchers began studying guaraná in the 1940s, finding that the native Indians' uses to cure fevers, headaches, cramps, and as an energy tonic were well founded. In the United States today, guaraná is reputed to increase mental alertness, fight fatigue, and increase stamina and physical endurance. Presently, guaraná is taken daily as a health tonic by millions of Brazilians, who believe it helps overcome heat fatigue, combats premature aging, detoxifies the blood, and is useful for intestinal gas, obesity, dyspepsia, fatigue, and arteriosclerosis. The plant, considered an adaptogen, is also used for heart problems, fever, headaches, migraine, neuralgia, and diarrhea.

In a study called, “Pharmacological activity of Guaraná (Paullinia cupana Mart.) in laboratory animals” researchers discovered that Mice that ingested a suspension of guaraná (Paullinia cupana, Sapindaceae) in a dose of 0.3 mg/ml showed a significant increase in physical capacity when subjected to a stressful situation such as forced swimming after 100 and 200 days of treatment. Additionally, guaraná, both after a single (3.0 and 30 mg/kg) or chronic administrations (0.3 mg/ml), was able to partially reverse the effects of amnesia as measured through a passive avoidance test in mice and rats, indicating a positive effect on memory acquisition.

There was also a tendency of rats treated with 0.3 mg/ml of guaraná to better maintain the memory of a Lashley III maze path. The animals all had the same average lifespan as those treated with a placebo, indicating a low toxicity of guaraná, even after 23 months of treatment.

A 2007 human pilot study assessed acute behavioral effects to four doses (37.5 mg, 75 mg, 150 mg and 300 mg) of guaraná extract. Memory, alertness and mood were increased by the two lower doses, confirming previous results of cognitive improvement following 75 mg guaraná.

In pharmacology and toxicology, a route of administration is the path by which a drug, fluid or other substance is brought into contact with the body. The pharmacokinetic properties of a substance (that is, those related to processes of uptake, distribution, and elimination) are critically influenced by the route of administration. Guaraná is currently available in many forms, such as chocolate bars, capsules, powder, syrup and energy drinks. All of these forms administer the guaraná in a conventional oral dosage form. Oral administration requires the guaraná to be swallowed, whereupon it is absorbed in the stomach and exposed to the hostile environment of the gastrointestinal tract and continues through the hepatic first pass metabolic processes in the liver before it is distributed to the rest of the body. This process determines the release rate, which will affect the guaraná's bioavailability and variability in absorption between different consumers.

In view of the foregoing, it will be appreciated that improvement in the methods of administering herbal supplements, such as guaraná, so as to increase the bioavailability and reduce variability in dosing would be highly desirable.

The above mentioned drawbacks of the traditional methods of delivery may be overcome through the sublingual and buccal administration of guaraná disclosed in the present invention. In this way the local component of the gastrointestinal damaging action is eliminated and the first massive passage through the liver is avoided providing for decreased variability in absorption and improved bioavailability.

The word “sublingual” literally means “under the tongue.” It refers to a method of administering substances in the mouth so that they can be rapidly absorbed into the blood vessels. The substance is absorbed through the buccal mucosa and into the sublingual vein where it has direct access to the blood circulation and is then carried throughout the whole body. Medical science has been using this method for years in the administration of cardiovascular drugs, steroids, and some barbiturates. The sublingual method has been life-saving for individuals who have had to rely on its speed and efficiency during times of critical emergency.

The principle behind sublingual administration is fairly simple. When a chemical comes in contact with the mucous membrane, or buccal mucosa, it diffuses into the epithelium beneath the tongue. This region contains a high density of blood vessels, and as a result, via diffusion, the substance quickly enters the venous circulation, which returns to the heart and then travels to the systemic arterial circulation. In contrast, substances absorbed by the bowel are subject to “first pass metabolism” in the liver before they are distributed to the rest of the body.

In theory, sublingual and buccal routes of administration have certain advantages over simple oral administration. This route is often faster, and entering a supplement into one's body sublingually ensures that the substance will only come in contact with the enzymes in saliva prior to entry into the bloodstream. Supplements otherwise orally administered must instead survive the extremely hostile environment of the gastrointestinal tract. This may mean a much greater percentage of the original substance is degraded either by the myriad of enzymes in the GI tract, such as monoamine oxidase, or the strong acids it contains. Additionally, after GI absorption, the supplement is sent to the liver where it may be extensively metabolized; this is known as the first pass effect of drug metabolism. Due to the degradative qualities of the stomach and intestine, or the solubility of the GI tract, the oral route is not the preferred route of administration for certain substances.

Alternative routes of administration that do not involve gastric absorption will by-pass first-pass metabolism in the liver. There are many such alternative routes, such as buccal and sublingual administration. Sublingual administration may comprise the consumer holding a guaraná composition or dosage form under their tongue while the supplement diffuses into the mouth, through the mucosa lining of the mouth, and from there into the bloodstream. “Buccal” means relating to the cheek. In buccal administration, the consumer holds the guaraná composition or dosage form between their cheek and gum instead of under the tongue. Like the other alternatives to gastric delivery, buccal and sublingual administration may conceivably raise the bioavailability of guaraná and other herbal supplements by avoiding first pass hepatic metabolism.

In view of the foregoing, it will be appreciated that improvements in the methods of administering herbal supplements such as guaraná so as to increase the bioavailability and reduce variability in dosing would be highly desirable.

### BRIEF SUMMARY OF THE INVENTION

In accordance with one method of the invention, the bioavailability of guaraná and other herbal extract formulations is increased by sublingual or buccal administration relative to administration of a comparable dosage of guaraná or herbal extract in a conventional enteral dosage form. The bioavailability of guaraná and other herbal extracts is highly variable from person to person when administered by conventional enteral dosage forms. In accordance with another method of the invention sublingual or buccal administration of guaraná and other herbal extract formulations reduces the inter-person variability of the bioavailability of extracts.

Guaraná and other herbal extract combinations especially formulated to enhance energy, heart health, memory, libido, weight management, mood, joint care, anti-oxidants and to remedy ailments such as headaches and allergies are provided by this invention as well.

Dosage forms especially adapted for sublingual and buccal administration of guaraná and other herbal extract combinations are provided by this invention as well.

One preferred formulation in accordance with the present invention is a liquid. It is further contemplated that the liquid is in the form of a spray or drops.

In another embodiment, the sublingual administration of guaraná and other herbal extract combinations may take the form of a paste or gel. The paste or gel would be applied between the cheek and gum or under the tongue. The viscosity of the paste or gel can be adjusted to allow for the retention under the tongue.

In other embodiments, the sublingual administration of guaraná and other herbal extract combinations may take the form of tablets, lozenges, pastilles, pills, viscous liquids, gum, patches and the like.

In another formulation in accordance with the present invention there is provided a hard, compressed, rapidly dissolving tablet adapted for direct sublingual dosing.

In yet another formulation the compressed rapidly dissolving tablet comprises effervescent agents. These effervescent agents allow enhanced adsorption of the guaraná across the mucosal membranes in the sublingual cavity.

Some of the dosage form embodiments may also include an acidulant that acidifies the pH in the local environment in the sublingual or buccal cavity to accelerate release of guaraná into the bloodstream. Yet further dosage form embodiments of this invention enable timed release of the guaraná that is slow enough to avoid accumulation of guaraná in the mouth, yet rapid enough to be acceptable to a consumer who holds the dosage form in the mouth while the guaraná is being released. Among these dosage forms is a liquid that congeals in the mouth and conforms to the space under the tongue or between the cheek and gum to provide a large contact surface area and comfortable feel.

In some embodiments of the present invention, it is contemplated that guaraná and other herbal extract combinations are combined with inactive ingredients. Such inactive ingredients may be necessary to add bulk to the preparation, to bind the preparation and to add color or flavor to the preparation.

It is also contemplated that the present invention comprises other active ingredients in addition to guaraná and other herbal extract combinations, which may be added to the preparation. Such added active ingredients may include acetaminophen, ibuprofen, naproxen, aspirin or other analgesics, which may augment the effectiveness of guaraná and other herbal extract combinations in alleviating or ameliorating migraines, headaches and other aches and pains.

In the foregoing description, the invention has been described with reference to specific exemplary embodiments thereof. It will, however, be evident that various modifications and changes may be made thereunto without departing from the broader spirit and scope of the invention as set forth in the appended claims. The specification is accordingly to be regarded in an illustrative rather than a restrictive sense.

The present invention is an improved composition and method of administering herbal supplements such as guaraná so as to increase the bioavailability and reduce variability in dosing over the prior art.

### BRIEF DESCRIPTION OF THE SEVERAL DRAWINGS

FIG. 1 is a flow chart of a method for the extracting and processing of the guaraná used in the multiple formulas in accordance with one embodiment of the present invention;

FIG. 2 is a flow chart of a method for preparing guaraná for sublingual administration in accordance with one embodiment of the present invention;

FIG. 3 is a flow chart of a method for preparing a carrier with guaraná for sublingual administration in accordance with one embodiment of the present invention;

FIG. 4 is a flow chart of a method for preparing a 100 ml carrier with guaraná in accordance with one embodiment of the present invention;

FIG. 5 is a flow chart of a method for combining liquid herbal extracts in accordance with one embodiment of the present invention;

FIG. 6 is a flow chart of a method for combining liquid herbal extracts and aromatic oils in accordance with one embodiment of the present invention; and

FIG. 7 is a flow chart of a method for combining liquid herbal extracts with a liposomal delivery vehicle in accordance with one embodiment of the present invention.

### DETAILED DESCRIPTION OF THE INVENTION

Sublingual and buccal administration of guaraná may improve the herbal supplement's bioavailability and greatly diminish absorption variability between consumers.

In general, the compositions of the present invention will be administered in a therapeutically effective amount by any accepted sublingual and buccal modes of administration. The guaraná may be present in the compositions and formulations in an amount sufficient to relieve, and/or inhibit conditions mentioned below. The therapeutic dosage of the compositions of the present invention will vary according to the particular use for which the treatment is made, the manner of administration of the compound and the health and condition of the consumer. Effective doses can be extrapolated from dose-response curves derived from in vitro or animal model test systems known in the art. Amounts effective for a particular use will depend on the age, weight and general condition of the consumer.

One aspect of the present invention is a method of administering herbal supplements by buccal or sublingual administration of guaraná and other herbal extract combinations. Guaraná can be administered in any composition or dosage form that can be held in the mouth for an extended period of time and permits diffusion or erosion of the drug into the mouth cavity where it can be absorbed through the mucosa lining of the mouth. Such dosage forms may include tablets, lozenges, pastilles, pills, viscous liquids, pastes, sprays, drops, gels, patches and the like.

Additional embodiments of the present invention also provide methods of administration, as well as a metered dosage system for administration of the guaraná spray and a liposomal delivery method.

One aspect of this invention is a method of increasing the bioavailability of guaraná by means of buccal or sublingual administration. Bioavailability refers to the proportion of the supplement administered that reaches the physiological site where the supplement exerts its therapeutic effect, and is generally regarded as the blood stream for most supplements. The bioavailability of a supplement administered in different formulations and by different routes can be compared using methods well known in the art. The bioavailability of a supplement is most readily expressed as the concentration of the supplement in the blood plasma integrated over time. This quantity is commonly referred to as the “area under the curve” or “AUC”. The bioavailability of a supplement administered in different formulations and by different routes can be compared by comparing the AUCs from consumers that have taken both formulations at different times. For a comparative study of different formulations on humans, a population of test subjects may be divided into two groups equal in number. Under controlled conditions, one group may be administered the supplement in one formulation while the other group is administered the other formulation. Their blood plasma concentrations of the supplement may be monitored for a period of time and the data collected and analyzed. A “wash out” period is then allowed to pass during which the supplement is eliminated from their bodies so that a second phase of the study may begin with a zero blood plasma concentration of the supplement. In the second phase, the group that received the first formulation of the supplement is administered the supplement in the second formulation, the group that received the second formulation is administered the first formulation, and monitoring, data collection and analysis are repeated. Administering both formulations to the same population minimizes error in comparison of bioavailability due to age, sex and individual physiological factors.

Buccal or sublingual administration of guaraná and other herbal extract combinations according to this invention preferably increases the extract's bioavailability as determined by comparison of the AUC of the particular composition or dosage form used with the AUC for users who swallow a conventional oral dosage form containing an equivalent dosage of guaraná. Alternatively, the increase in bioavailability can be determined against a dosage form of different strength provided the difference is taken into account.

Guaraná has practically the same chemical composition as caffeine and has the same physiological actions, thus its use for mental fatigue and heat exhaustion. It contains up to 7% of guaranine or caffeine (as compared to about 2% in coffee), with theobromine, theophylline, xanthine, and other xanthine derivatives, as well as an appreciable amount of tannins (approximately 12%, including d-catechin), and saponins, starch, fats, choline, and pigments.

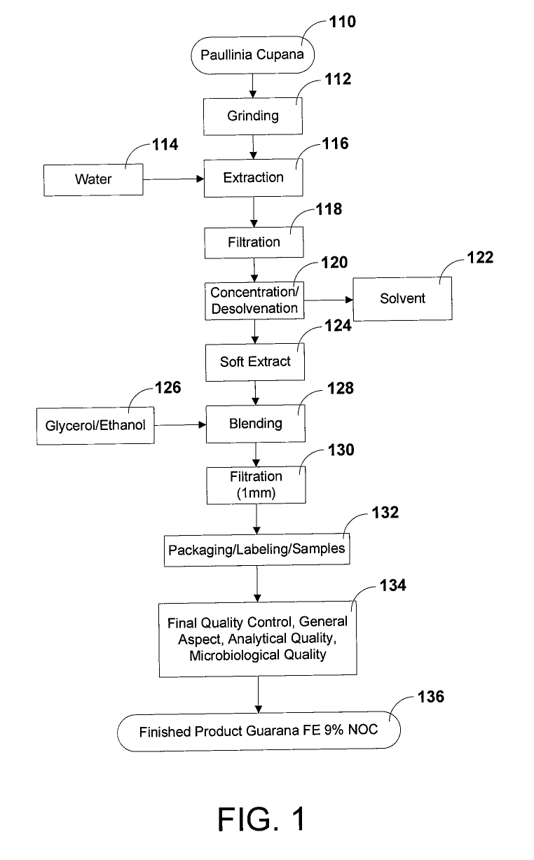

Some embodiments of the present invention, described with reference to FIG. 1, may comprise a method of using an herbal extraction plant for extracting guaraná from the Paullinia Cupana plant. In some embodiments a solvent extraction method may be used with a polar solvent such as water, methanol, ethanol or any mixture thereof, or any non-polar solvent, to achieve a low temperature and short contact process which allows for extracting the product with maximum recovery of the active ingredient since thermal degradation is avoided. One embodiment of the present invention may comprise harvesting berries of the Paullinia Cupana plant 10, grinding 12 the dried berries into guaraná seed powder and sending the dried powder to an extracting vessel where water may be added 14 to the guaraná powder to extract 16 the guaraná. In some embodiments the water 14 will be kept at low temperatures, and the extraction 16 completed in a short time, to reduce any degeneration of the guaraná. The extracted guaraná may then be delivered to a continuous belt filter press or other type filter for filtering 18 and then sent to a solvent extractor to perform a concentration/desolvenation 20 process on the filtered extract by removing the solvent 22 from the mixture in the solvent extractor which may be a counter current or continuous percolation type extractor. This type of desolventizing unit may use indirect heat to remove significant amount of the solvent from the residue to produce a soft extract 24 solution. Direct steam may be employed to remove trace amounts of solvent remaining. The vapors from the desolventizer may be fed to the distillation column(s), otherwise they may be recycled back to the extractor.

In some embodiments of the present invention a glycerol/ethanol mix may be added 26 to the soft extract 24 and the two substances may be blended thoroughly and then passed through a 1 mm filtration process 30 to achieve the final clarified guaraná extract product. The extract may then be packaged and labeled 32 and samples taken for final quality control 34. The finished guaraná extract 36 will then be ready for market.

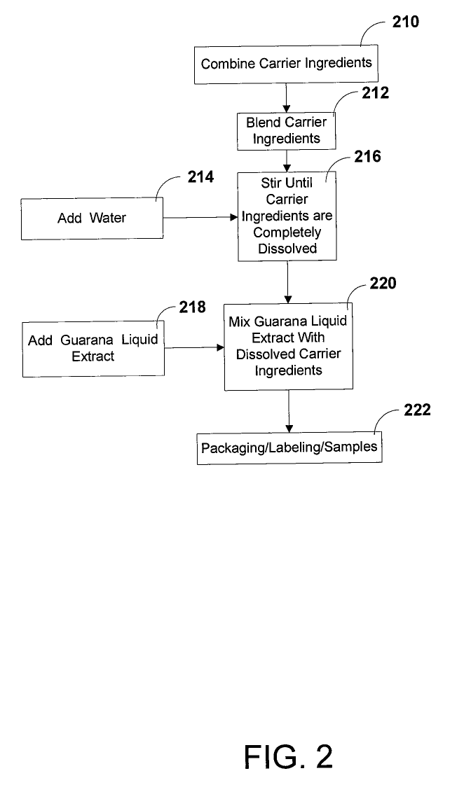

Further embodiments of the present invention, described with reference to FIG. 2, may comprise combining 210 various carrier ingredients and blending 212 the carrier ingredients thoroughly using methods known in the art. The carrier ingredients may comprise any number of ingredients known in the art for use in buccal and sublingual administration. In some embodiments, water may then be added 214 to the blended carrier ingredients and the water and carrier ingredients may then be stirred 216 until the carrier ingredients are completely dissolved. The water may be preserved water, purified water, distilled water or any other treated water suitable for the purpose. In some embodiments the water may be heated to a temperature of between 80-90 degrees Celsius (below the boiling point) to improve the dissolving process while reducing the degeneration of the blended ingredients as much as possible.

Once the carrier ingredients are completely dissolved in the water, a guaraná liquid extract may then be added 218 to the carrier solution and mixed 220 thoroughly. The finished product may then be packaged 222, labeled and samples taken for quality assurance purposes. In some embodiments the finished product may be packaged in oral syringes for convenient application directly onto the tissue under the consumer's tongue.

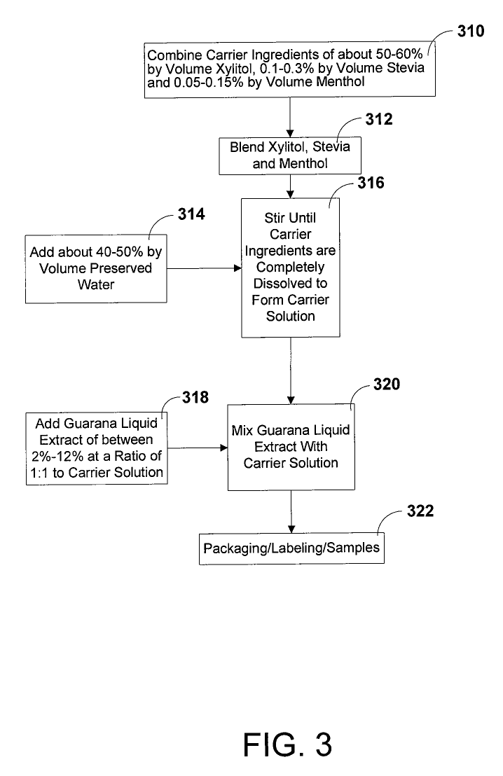

Further embodiments of the present invention, described with reference to FIG. 3, may comprise combining 310 carrier ingredients of about 50%-60% by volume Xylitol, 0.1-0.3% by volume Stevia and 0.05-0.15% by volume Menthol and blending 312 together the carrier ingredients using methods known in the art.

In some embodiments, from about 40%-50% by volume of “preserved water” which has been preheated to between 80-90 degrees Celsius may then be added 314 to the blended carrier ingredients and the heated preserved water and carrier ingredients may then be stirred 316 until the carrier ingredients are completely dissolved to form a carrier solution.

Preserved water may comprise water containing a known preservative to limit the growth of bacteria and other microorganisms that can consume or adversely affect the potency of a remedy. In some embodiments preserved water may be prepared with 1.9 grams Methylparaben NF and 0.96 grams propylparaben NF which may be dissolved in 200 ml distilled water and made up with distilled water to a volume of 1 gallon. In some embodiments preserved water may comprise purified water with a ratio of 0.5% Methylparaben and 0.25% Propylparaben. In some embodiments the water may be heated to a temperature of between 80-90 degrees Celsius (below the boiling point) to improve the dissolving process while reducing the degeneration of the blended ingredients as much as possible.

Once the carrier ingredients are completely dissolved in the preserved water and the solution has been allowed to cool, a guaraná liquid extract of between 2% to 12% may then be added 318 to the carrier solution at a ratio of about 1:1 and mixed 320 thoroughly. The finished product may then be packaged 322, labeled and samples taken for quality assurance purposes.

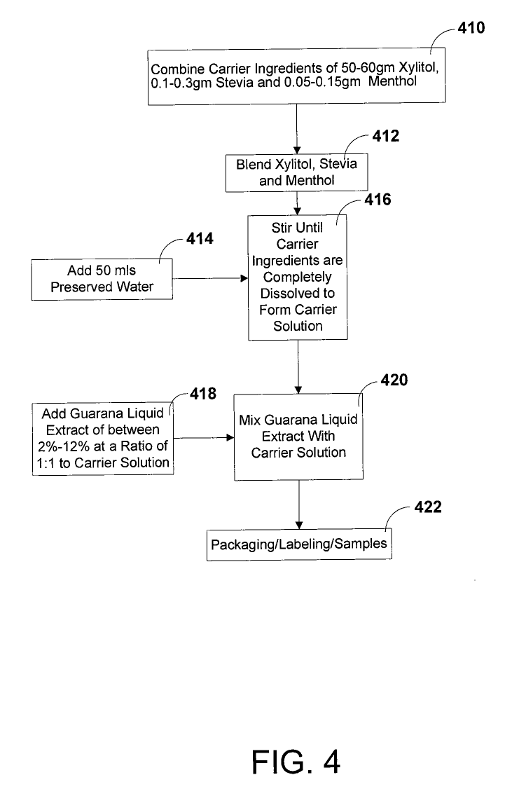

Further embodiments of the present invention, described with reference to FIG. 4, may comprise combining 410 50-60 gm Xylitol with 0.1-0.3 gm Stevia and 0.05-0.15 gm Menthol. and blending 412 together the carrier ingredients using methods known in the art.

In some embodiments, 50 mls of “preserved water” which has been preheated to between 80-90 degrees Celsius may then be added 414 to the blended carrier ingredients and the heated preserved water and carrier ingredients may then be stirred 416 until the carrier ingredients are completely dissolved to form a carrier solution.

Once the carrier ingredients are completely dissolved in the preserved water and the solution has been allowed to cool, a guaraná liquid extract of between 2% to 12% may then be added 522 to the carrier solution at a ratio of about 1:1 and mixed 420 thoroughly. The finished product may then be packaged 422, labeled and samples taken for quality assurance purposes.

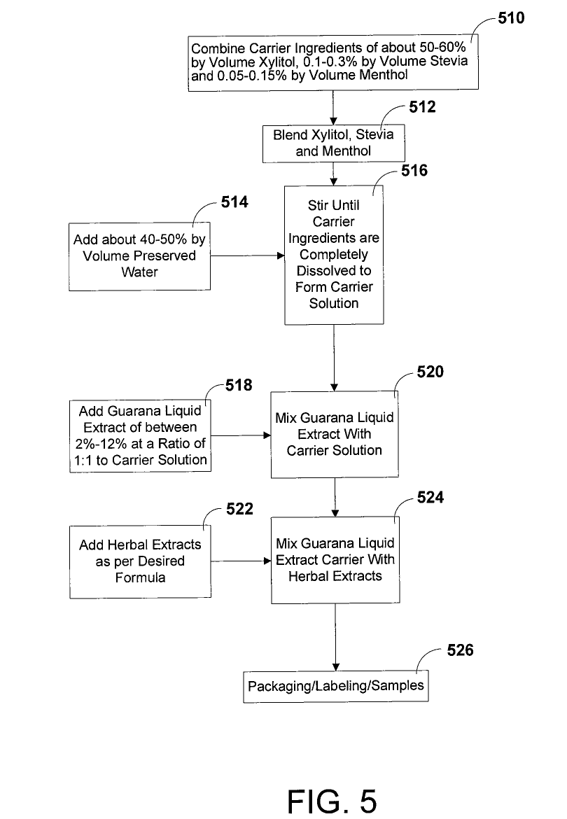

Further embodiments of the present invention, described with reference to FIG. 5, may comprise combining 510 carrier ingredients of about 50%-60% by volume Xylitol, 0.1-0.3% by volume Stevia and 0.05-0.15% by volume Menthol and blending 512 together the Xyltol, Stevia and Menthol using methods known in the art.

In some embodiments, from about 40%-50% of “preserved water” which has been preheated to between 80-90 degrees Celsius may then be added 514 to the blended carrier ingredients and the heated preserved water and carrier ingredients may then be stirred 516 until the carrier ingredients are completely dissolved forming a carrier solution.

Once the carrier ingredients are completely dissolved in the preserved water and the solution has been allowed to cool, a guaraná liquid extract of between 2% to 12% may then be added 522 to the carrier solution at a ratio of about 1:1 and mixed 520 thoroughly.

In some embodiments, once the guaraná liquid extract and carrier solution are mixed thoroughly, a formula of herbal extracts may be added 522 to the solution depending on the remedy sought or physical enhancement desired.

One embodiment of the present invention may comprise a remedy for fatigue and provide for increased energy and physical performance. The embodiment may add 522 the extracts of Vitamin B-12, Vitamin C, Vitamin E, Folic Acid, Capsaicin, Ginseng, Coenzyme Q10 (CoQ10), Magnesium, Zinc, Potassium, Niacin, Thiamin and Avena Sativa.

Vitamin B-12 is important for the normal functioning of the brain and nervous system, and for the formation of blood. Vitamin C is an essential nutrient and is required for a range of essential metabolic reactions in humans. Vitamin C is also a highly effective antioxidant, since it protects the body against oxidative stress, and is a cofactor in several vital enzymatic reactions. People consuming diets rich in vitamin C are healthier and have lower mortality from a number of chronic illnesses. Vitamin E is a fat-soluble vitamin with antioxidant properties and is believed to protect cell membranes. Folic Acid (also known as Vitamin M and Folacin) are forms of the water-soluble Vitamin B9 and is necessary for the production and maintenance of new cells. Capsaicin is the active component of chili peppers. It is common for people to experience pleasurable and even euphoriant effects from eating capsaicin-flavored foods. This has been attributed to pain-stimulated release of endorphins. Ginseng roots are taken orally as adaptogens, a product that increases the body's resistance to stress. Coenzyme Q10 (CoQ10) is an oil-soluble vitamin-like substance which is a component of the electron transport chain and participates in aerobic cellular respiration, generating energy in the form of ATP. Ninety-five percent of the human body's energy is generated this way. CoQ10 has the ability to transfer electrons and therefore act as an antioxidant. Magnesium ions are essential to the basic nucleic acid chemistry of life, and thus are essential to all cells of all known living organisms. Many enzymes require the presence of magnesium ions for their catalytic action, especially enzymes utilizing ATP, or those which use other nucleotides to synthesize DNA and RNA. Magnesium is a vital component of a healthy human diet. Zinc supports healthy adrenal activity which combats the negative effects of stress and translates into more energy. Zinc has also been shown to boost the immune system. Potassium is important in neuron (brain and nerve) function, and in influencing osmotic balance between cells and the interstitial fluid. Potassium is also important in allowing muscle contraction and the sending of nerve impulses. Niacin, also known as vitamin B3, is a water-soluble vitamin shown to speed wound healing and help the immune system fight off viral infections. Thiamin or thiamine, also known as vitamin B1 It is essential for neural function and carbohydrate metabolism. Avena sativa is what most people know as oats. Oats have been used for medical purposes since the Middle Ages and are known to increase energy levels.

In one preferred embodiment of the invention, the composition is comprised of the above herbal ingredients in the following approximate proportions relative to each other: about 0.01%-10% by volume Vitamin B-12, Vitamin C, Vitamin E, Folic Acid, Capsaicin, Ginseng, Coenzyme Q10 (CoQ10), Magnesium, Zinc, Potassium, Niacin, Thiamin and Avena sativa extracts. However, it should be understood that the invention is not limited in this regard, and the relative amounts of these ingredients can be varied to achieve similar results.

Another embodiment of the present invention provides for improved heart health. The embodiment may add 522 the extracts of Vitamin B3, Vitamin B6, Vitamin B12, Hawthorn, Ginseng, Cayenne, Bilberry, Garlic, Evening Primrose Oil (EPO), Coleus Forskohlii and Sage.

Hawthorn is a cardio (heart) tonic and is used for its stimulating and sedating properties. Hawthorn increases blood flow to the heart by dilating the coronary arteries, lowers blood pressure and eases the heart's workload by dilating arteries in the arms and legs, and increases the force of the heart's contractions. It strengthens the force of each contraction of the heart and slows the heart rate down when necessary. Ginseng (Ginsana) is an herbal remedy to help fight fatigue, improve performance, and fight off stress. Cayenne has anti-inflammatory, antioxidant, antiseptic, diuretic, analgesic, expectorant, and diaphoretic properties. Cayenne is used worldwide to treat a variety of health conditions, including heart disease. Cayenne stimulates blood flow, strengthening the heart, arteries, capillaries and nerves. Bilberry is the small dark fruit of a deciduous shrub native to Europe, akin to the North American blueberry. Bilberries contain powerful antioxidants that neutralize potentially dangerous free radicals in the body. Bilberry has been used to treat angina. The flavonoids found in bilberries thin the blood and prevent fragility of the capillaries. Garlic is promoted to reduce cholesterol and prevent hardening of the arteries. Evening primrose seed oil (EPO) is used to prevent Heart Disease and lower blood pressure. Coleus Forskohlii is the source of a unique substance known as forskolin. Forskolin helps naturally increase a signaling molecule in bodies called cyclic adenosine monophosphate, or cAMP. In turn, cAMP supports blood vessel relaxation and healthy heart muscle contractions. Sage has long been used for its effect on the Medulla oblongata (brain stalk) and provides remedial action for breathing, heart, and blood pressure problems.

In one preferred embodiment of the invention, the composition is comprised of the above mentioned herbal ingredients in the following approximate proportions relative to each other: about 0.01%-10% by volume Vitamin B3, Vitamin B6, Vitamin B12, Hawthorn, Ginseng, Cayenne, Bilberry, Garlic, Evening Primrose Oil (EPO), Coleus Forskohlii and Sage extracts. However, it should be understood that the invention is not limited in this regard, and the relative amounts of these ingredients can be varied to achieve similar results.

Yet another embodiment of the present invention may provide for improved mental alertness and memory enhancement. The embodiment may add 522 extracts of Ginko Bilbao, Gotu Kola, CoQ10, Vitamin E, Vitamin C, Folate, Ginseng, Lemon balm and Theobroma cacao. Ginkgo contains numerous antioxidants such as the proanthocyanidins, flavonoids, that counteract free-radical activity. Flavonoids are also known to strengthen capillaries, which can promote healthy blood flow to the brain, helping to maintain cognitive health and improve memory. Gotu Kola is a member of the carrot family native to Madagascar, India and Sri Lanka. Gotu kola has been shown to improve circulation in the brain, contributing to enhanced mental performance and memory retention. Folate is a water-soluble B vitamin that occurs naturally in food. Folic acid is the synthetic form of folate and helps produce and maintain new cells. Ginseng (Ginsana) is an herbal remedy to help fight fatigue, improve performance, and fight off stress. Lemon balm, also known as Melissa officinalis, is a traditional herbal medicine widely known to possess memory or cognition-enhancing properties.

Theobroma Cacao is cultivated in South and Central America and it is reported that the flavanoids found in Cacao are beneficial to vascular health and can aid blood circulation to the brain and improve memory.

In one preferred embodiment of the present invention, the composition is comprised of the above mentioned herbal ingredients in the following approximate proportions relative to each other: about 0.01%-10% by volume Ginko Bilbao, Gotu Kola, CoQ10, Vitamin E, Vitamin C, Folate Ginseng, Lemon balm and Theobroma cacao extracts. However, it should be understood that the invention is not limited in this regard, and the relative amounts of these ingredients can be varied to achieve similar results.

Yet another embodiment of the present invention may provide for a remedy for headaches. The embodiment may add 522 the extracts of White Willow Bark, Feverfew, Lavender, Cat's Claw, Vitamin C, Gingko, Ginseng, Rosemary puree, Milk Thistle and generic analgesics.

White Willow Bark is a tree native to Europe and Asia. White willow bark has been shown to be more effective than aspirin and not to be as irritating to the stomach lining because of compounds that are found in the bark. Feverfew (Tanacetum Parthenium) has been shown to reduce the frequency and severity of migraines, as well as frequently associated symptoms of nausea and vomiting. Lavender has long been used as a remedy due to its calming, soothing, and sedative effects. Cat's claw is a woody climbing plant native to the Amazon rainforest that grows throughout the tropical jungles of Central and South America and has been used by the Asháninka tribe of Peru for over 2,000 years to treat diverse health complaints including headache. Vitamin C is an essential nutrient and is required for a range of essential metabolic reactions in humans. Vitamin C is also a highly effective antioxidant, since it protects the body against oxidative stress, and is a cofactor in several vital enzymatic reactions. For nearly 2800 years the Chinese have used extracts from the ginkgo bilbao tree as natural medicine to treat a variety of conditions. Ginkgo contains numerous antioxidants such as the proanthocyanidins, flavonoids, that counteract free-radical activity. Flavonoids are also known to strengthen capillaries, which can promote healthy blood flow to the brain, helping to maintain cognitive health reduce headaches. Ginseng (Ginsana) is a dietary supplement that is being promoted as an herbal adaptogen, which helps the user adapt to physical or emotional stress and fatigue. Rosemary is a circulatory and nervine stimulant which may be used whenever psychological tension is present and is used effectively for headaches, particularly migraines. Milk thistle (Silybum marianum) has been used since Greco-Roman times as an herbal remedy for a variety of ailments. A flavonoid complex called silymarin can be extracted from the seeds of milk thistle and is believed to be the biologically active component. The terms “milk thistle” and “silymarin” are often used interchangeably. Milk thistle has been known to help decrease or eliminate migraine headaches.

In one preferred embodiment of the present invention, the composition is comprised of the above mentioned herbal ingredients in the following approximate proportions relative to each other: about 0.01%-10% by volume White Willow Bark, Feverfew, Lavender, Cat's Claw, Vitamin C, Gingko, Ginseng, Rosemary puree, Milk Thistle extracts and generic analgesics. However, it should be understood that the invention is not limited in this regard, and the relative amounts of these ingredients can be varied to achieve similar results.

Yet another embodiment of the present invention may provide for a libido enhancer. The embodiment may add 522 the extracts of Vitamin B, Ginko, Horny Goat Weed, Cnidium, Cistanche Bark, Vitamin A & E, Selenium, Zinc, Magnesium, Maca, Damiana and Catuaba Bark.

B Vitamins enhance the brain's signals to your glands to initiate the hormone production and flow of blood to the sex organs. B-Vitamins are critical to the development of brain messengers for these signals. Vitamin B-6 is especially important because it controls elevated prolactin, a libido saboteur. It also monitors the body's balance between estrogen and progesterone, reducing excess estrogen, which can cause severe PMS or perimenopausal mood swings in women. Ginkgo Bilbao is an herb that can improve sexual function in men. Gingko Bilbao is used to improve blood flow around the body including to the genitals and also functions as an anti-oxidant in the body. Ginkgo has long been thought to heal male impotence, and is a standard herbal remedy prescribed in China and is now popular worldwide. It helps to prevent lipid per oxidation of cell membranes, which is the process that leads to clogging of the arteries, or atherosclerosis, which are vital for blood flow to the penis. Horny Goat Weed has been used by Chinese herbalists to improve sexual functions in both men and women. Horny goat weed stands as a time-tested aphrodisiac that increases libido in men and women, and improves erectile function in men. After centuries of use in China, top medical doctors now report that it can be used to boost libido, improve erectile function, restore sexual power and increase sensation. It works by freeing up testosterone, which naturally increases sex drive and endurance. Cnidium is known in China as She Chuang Zi and is a popular herb worldwide renowned for its health benefits and acts as a sexual tonic. Cnidium works in a similar way to Viagra to increase nitric oxide release and inhibit PDE-5. This enables an erection to be maintained for longer period of time. Cnidium also assists in maintaining healthy blood circulation throughout the body. Cistanche Bark is an important medicinal plant in traditional Chinese medicine. It is a tonic herb which increases the blood circulation. Cistanche is used for increasing energy and to reinforce the vital function of the sexual organs and induce Taxation, for the treatment of impotence, low sex drive and premature ejaculation. Selenium is believed to be good for sperm motility and mobility, nearly 50% of the selenium in a man is in the testicles and seminal ducts; men lose selenium in their semen therefore getting enough selenium is vital for peak sexual performance. Zinc is needed for the production of testosterone. Zinc content of the prostate gland and sperm is higher than in any other part of the body. A lack of zinc is associated with sexual problems, including sperm abnormalities and prostate disease. Zinc helps produce testosterone, but also helps to maintain semen volume and adequate levels of testosterone, thus maintaining sex drive. Magnesium is important for the production of sex hormones, including androgen and estrogen and neurotransmitters from the brain that modulate sex drive such as dopamine and norepinephrine. A Magnesium deficiency will decrease oxygen delivery to your muscle tissue. Magnesium promotes muscle strength and endurance. Maca root, from the South American country of Peru, has been passed down from the Inca and is taken to increase strength, energy, stamina, libido and sexual function. Damiana (Turnera Aphrodisiaca) is renowned for its libido enhancing qualities. Damiana produces a feeling of mild euphoria which relaxes the body and calms the mind. The herb also helps to balance female hormone levels and leads to increased libido in females. Catuaba bark comes from the catuaba tree which grows in the forests of northern Brazil (its scientific name is Erythroxylum catuaba) and is widely known to enhance libido and as a remedy for impotency.

In one preferred embodiment of the present invention, the composition is comprised of the above mentioned herbal ingredients in the following approximate proportions relative to each other: about 0.01%-10% by volume Vitamin B, Ginko, Horny Goat Weed, Cnidium, Cistanche Bark, Vitamin A & E, Selenium, Zinc Magnesium, Maca and Damiana extracts. However, it should be understood that the invention is not limited in this regard, and the relative amounts of these ingredients can be varied to achieve similar results.

Yet another embodiment of the present invention may provide for a weight loss enhancer. The embodiment may add 522 the extracts of Capsaicin, Ginseng, Hoodia Gordonii, Vitamin B12, Vitamin C, Vitamin E, Garcinia cambogia and Cereus grandiflorus.

Capsaicin is the active component of chili peppers. Studies show that Capsaicin can prevent new fat cells from forming as well as decrease the appetite and reduce fat in the body. One study revealed that the subjects who took the capsaicin experienced a significant increase in their mean metabolic rate at 30 minutes and 60 minutes after taking the extract. Throughout the study that ranged a two week period, body fat was reduced in 70% of the subjects who had taken the capsaicin extract. Ginseng (Ginsana) is an adaptogen which helps one adapt to physical or emotional stress and fatigue. A study from the University of Chicago using obese diabetic mice reported that an extract from American ginseng berry may reduce blood sugar levels by 30 percent and aid weight loss.

Hoodia (pronounced HOO-dee-ah) is a cactus-like plant that grows primarily in the semi-deserts of South Africa, Botswana, Namibia, and Angola. Scientists believe that the active ingredient in Hoodia, P57, acts on the brain in a manner similar to glucose and sends the message that you are full even when you have not eaten, thus decreasing your desire to eat. In one study, obese volunteers who took Hoodia ended up eating about 1,000 calories per day less than those who did not take the supplement. Vitamin B-12 is one of eight B vitamins which is important for the normal functioning of the brain and nervous system, and for the formation of blood. It is normally involved in the metabolism of every cell of the body, especially affecting DNA synthesis and regulation, but also fatty acid synthesis and energy production. Vitamin C is an essential nutrient and is required for a range of essential metabolic reactions in humans. Vitamin C is also a highly effective antioxidant, since it protects the body against oxidative stress, and is a cofactor in several vital enzymatic reactions. Vitamin E is a fat-soluble vitamin with antioxidant properties and is believed to protect cell membranes. Garcinia cambogia, also called Malabar tamarind, has been used many centuries in Southern India. The principal acid of garcinia fruit, hydroxycitrate, has been shown to curb appetite and inhibit fatty acid synthesis (production) in animal studies. Cereus grandiflorus is a night-blooming cactus species originating from the Antilles, Mexico and Central America that has been studied for its qualities as an appetite suppressant.

In one preferred embodiment of the present invention, the composition is comprised of the above mentioned herbal ingredients in the following approximate proportions relative to each other: about 0.01%-10% by volume Capsaicin, Ginseng, Hoodia Gordonii, Vitamin B12, Vitamin C, Vitamin E, Garcinia cambogia and Cereus grandiflorus extracts. However, it should be understood that the invention is not limited in this regard, and the relative amounts of these ingredients can be varied to achieve similar results.

Another embodiment of the present invention may provide for a mood enhancement formula. The embodiment may add 522 the extracts of Lithium, Skullcap, Valerian, Passion Flower, Polygala, Vitamin E, Hawthorne, Ginko, Cyracos, Passion Flower and Kava.

Lithium is one of the most common elements used for stabilizing mood swings, mania and depression. Lithium orotate is a highly bioavailable form of lithium that is available without a prescription. Because of its superior bioavailability, (20 times more bio-active than other lithium salts) lower doses of lithium orotate (Rather than lithium carbonate or lithium citrate) may be used to achieve therapeutic brain lithium concentrations and relatively stable serum concentrations. Skullcap relaxes states of nervous tension while at the same time renewing and revivifying the central nervous system. Skullcap is used by some herbalists as a treatment for anxiety, stress and tension. It is said to aid in preventing panic attacks. Taken at bedtime, it can promote sleep. Research has found that Valerian may be effective for insomnia, anxiety and restlessness. It is considered safe and is not habit-forming. Studies have shown that it can help you get to sleep quicker and sleep better without next-day drowsiness. Passion Flower is a woody vine that bears small berry-like fruit called grandilla. Passion Flower has been used to relax the Central Nervous System (CNS) and promote emotional balance. In clinical study, the active components of Passion Flower have been shown to provide positive support for occasional nervousness, nervous tension, and anxiety depressed mood and mild to moderate mood changes caused by everyday stress Restlessness and occasional sleep difficulties. Polygala smallii is a short-lived, herbaceous member of the milkwort family. In traditional Chinese medicine, other varieties of polygala are used for a variety of purposes, including the promotion of sleep and calming the spirit. Polygala is considered a powerful tonic herb that can help develop the mind and aid in creative thinking. Vitamin E is a fat-soluble vitamin with antioxidant properties and is believed to protect cell membranes. Hawthorne (Crataegus oxyacantha) is native to Europe with close species found in North Africa and western Asia. The tree has been known and appreciated throughout the ages, by the ancient Greeks, Arabs, and Europeans. Western herbalists consider it an excellent relaxant. Ginko (Ginkgo biloba) has long been used as a medicine in its native China and it has now been shown that ginkgo has a profound activity on brain function and cerebral circulation which is useful to prevent depression and other symptoms related to poor brain circulation. Cyracos lemon balm extract is prepared from special lemon balm chosen for its high concentrations of hydroxycinnamic and rosmarinic acids. These active lemon balm constituents appear to enhance mood and cognitive performance by exhibiting central nervous system acetylcholine receptor activity, including nicotinic and muscarinic binding properties in the cerebral cortex of the human brain. Passion Flower (Passiflora incarnata) is from North America. The aerial (above ground) portions of the Passion Flower vine are used to derive medicinal compounds that relax the Central Nervous System (CNS) and promote emotional balance. In clinical studys, the active components of Passion Flower have been shown to provide positive support for occasional nervousness, nervous tension, anxiety, depressed mood and mild to moderate mood changes caused by everyday stress. Kava Kava is a member of the pepper family which grows as a bush in the South Pacific. Captain James Cook first discovered kava, and gave the plant the botanical name which means intoxicating pepper. Kava has been used for thousands of years for its medicinal effects as a remedy for nervousness and insomnia. Recent clinical studies have shown that kava is a safe, non addictive anti-anxiety herbal extract.

In one preferred embodiment of the present invention, the composition is comprised of the above mentioned herbal ingredients in the following approximate proportions relative to each other: about 0.01%-10% by volume Lithium, Skullcap, Valerian, Passion Flower, Polygala, Vitamin E, Hawthorne, Ginko, Cyracos, Passion Flower and Kava extracts. However, it should be understood that the invention is not limited in this regard, and the relative amounts of these ingredients can be varied to achieve similar results.

Another embodiment of the present invention may provide for a joint care formula. The embodiment may add 522 the extracts of MSM, Selenium, Glucosamine HCL, Yucca, Chondroitin, Vitamin E, Boswellia Serrata, White Willow, Feverfew, Devil's Claw and Turmeric.

Methylsulfonylmethane (MSM) contains sulfur that can be utilized by the body in the formation of connective tissue, such as articular cartilage. Arthritic joints have been found to be low in both sulfur and cysteine. A recent study on MSM found that it had a significantly beneficial effect on joint pain and stiffness. Those taking the MSM supplement had statistically significant lower levels of the amino acid homocysteine in their blood. Homocysteine is believed to cause damage to the lining of the arteries, increase clotting in the blood, and when it is present in high levels, causes blockages in the arteries. Selenium is a trace mineral that is essential to good health but required only in small amounts. Selenium is incorporated into proteins to make selenoproteins, which are important antioxidant enzymes. The antioxidant properties of selenoproteins help prevent cellular damage from free radicals. Glucosamine HCL, which is produced in the body, is a precursor molecule in the synthesis of proteoglycans and glycosaminoglycans (GAGs). These proteins are the structural components of cartilage—the shock absorber that gives joints strength and resilience. Glucosamine has demonstrated in a number of clinical trials to decrease articular pain, decrease joint tenderness, decrease swelling, and improve range of motion to a significant degree. Yucca stalk is reputed in western herbal tradition to support joints and blood sugar problems. The beneficial saponins are found in the Yucca plant's stalk and root. Yucca is also said to reduce joint inflammation.

Chondroitin is a unique nutrient used by your body to help keep your joints flexible and cushioned. It's comprised of collagen, fluid and other substances. Because it's a living tissue, it can regrow to help keep you flexible. Vitamin E stimulates cartilage synthesis and inhibits the breakdown of cartilage. Boswellia Serrata is a disease modifying agent and is beneficial in chronic inflammatory conditions. Boswellia Serrata reduces joint pain and swelling and increases mobility. It is safe in long term therapy and does not show any gastrointestinal side effects. White Willow Bark is an anti inflammatory herb which can help reduce swelling and pain in the joints. Feverfew is indigenous to Europe and the Balkan Peninsula and is said to have grown around the Greek Parthenon. Feverfew leaves are a known herbal remedy used to soothe joint inflammation and more. Devil's Claw has been shown to improve joint mobility and reduce the pain and swelling associated with arthritis. Devil's claw is recommended by natural health practitioners for osteoarthritis, rheumatoid arthritis, gout, muscle injury, joint injury and other inflammatory conditions. Turmeric (Curcuma longa) is native to India and southern Asia where it is extensively cultivated. Turmeric contains an anti-inflammatory ingredient known to fight arthritis.

In one preferred embodiment of the present invention, the composition is comprised of the above mentioned herbal ingredients in the following approximate proportions relative to each other: about 0.01%-10% by volume MSM, Selenium, Glucosamine HCL, Yucca, Chondroitin, Vitamin E, Boswellia Serrata, White Willow, Feverfew, Devil's Claw and Turmeric extracts. However, it should be understood that the invention is not limited in this regard, and the relative amounts of these ingredients can be varied to achieve similar results.

Another embodiment of the present invention may provide for an allergy control formula. The embodiment may add 522 the extracts of Golden Seal, Nettle, Ginkgo, Vitamin C, Barberry, Echinacea, Eucalyptus, Linden, Perilla and Butterbur.

Goldenseal Root contains berberine which is an anti-bacterial and anti-fungal and is known to soothe inflamed mucous membranes. When used topically, Goldenseal provides and antibiotic effect. Nettle contains a naturally occurring antihistamine, Naturopathic. The stinging nettle has particular good results for those with a pollen allergy. In studies done using Nettle, significant relief from hay fever has been reported and it is a very effective part of any natural allergy treatment. Gingko Bilbao contains “ginkogolides.” These rarely spoken about substances can stop or limit allergy attacks. Vitamin C can have a dramatic effect in improving allergy symptoms, particularly hay fever and asthma, due to its ability to counteract the inflammation responses that are part of such conditions. Extracts from Barberry fruit appear to have natural antihistamine and anti-allergy potential. Echinacea is a natural remedy for sinus allergies and works by boosting the immune system. Eucalyptus is used to clear nasal passages, loosen phlegm, and increase flow of blood to muscles. It has antiseptic properties that are helpful for allergies, colds, flu, and sore throats. Eucalyptus is also a strong expectorant, suitable for chest infections, including bronchitis and pneumonia. Linden flowers, leaves and wood are used for medicinal purposes. Active ingredients in the linden flowers include flavonoids (which act as antioxidants), volatile oil, and mucilage components (which are soothing and reduce inflammation). The plant also been used in natural Hay fever remedies. Perilla is a genus of an annual herb that is a member of the mint family, Lamiaceae and its constituent rosmarinic acid has been suggested to have anti-allergic activity. Butterbur is a plant native to Europe, Africa, and Asia. Extracts from the roots, leaves, and flowers have been used traditionally to treat headaches, and coughs due to asthma, allergies, and infections. Studies have found that an extract from butterbur root can reduce the symptoms of asthma and allergies and that an extract of butterbur leaf works as well as an antihistamine in reducing allergy symptoms.

In one preferred embodiment of the present invention, the composition is comprised of the above mentioned herbal ingredients in the following approximate proportions relative to each other: about 0.01%-10% by volume Golden Seal, Nettle, Ginkgo, Vitamin C, Barberry, Echinacea, Eucalyptus, Linden, Perilla and Butterbur extracts. However, it should be understood that the invention is not limited in this regard, and the relative amounts of these ingredients can be varied to achieve similar results.

Another embodiment of the present invention may provide for an anti-oxidant formula. The embodiment may add 522 the extracts of Vitamin B3-6-12, Vitamin C, Vitamin E, CoQ10, Acai, Rosmarinic acid from Oregano, Peppermint, Lemon Balm, Lutein, Selenium and Bilberry.

Vitamins B3, B6 and B12 are necessary for normal breakdown of fats and fatty acids and the release of energy from carbohydrates. Vitamin B12 (cyanocobalamin) is an oxygen carrier; it decreases blood cholesterol and metabolizes fat. Vitamin B 12 is essential in humans for healthy nerve tissues. Vitamin C helps some of our most important body systems. First and foremost, it helps the immune system to fight off foreign invaders and tumor cells. Vitamin C also supports the cardiovascular system by facilitating fat metabolism and protecting tissues from free radical damage, and it assists the nervous system by converting certain amino acids into neurotransmitters. Vitamin E is a fat-soluble vitamin and an antioxidant that blocks some of the damage caused by toxic by-products released when the body transforms food into energy or fights off infection. CoQ10 is also a powerful antioxidant, and protects the body from free radical damage. Acai berry has 15-20 times the antioxidants (anthocyanins) that red grapes have and extracts of Acai seeds were reported to have antioxidant capacity against peroxyl radicals, peroxynitrite and hydroxyl radicals. Rosmarinic acid from Oregano (Origanum vulgare L.) is rich in phenolic compounds with high antioxidant and antimicrobial activity. Peppermint also contains the substance rosmarinic acid, which has antioxidant abilities to neutralize free radicals. Extracts of peppermint have also been shown to help relieve the nasal symptoms of allergic rhinitis (colds related to allergy). Lemon balm (Melissa officinalis), a member of the mint family, and laboratory studies suggest that lemon balm has antioxidant properties. Lutein is a carotenoid, a natural antioxidant and a free radical scavenger found in highest levels in the eye. The eye is continually subjected to oxidative stress due to light exposure and retinal metabolism, and research indicates that Lutein's chemical properties may retard the onset of degenerative and harmful effects in the eye. Selenium is an essential constituent of a number of enzymes with antioxidant functions. A deficiency of Selenium has been reported to make humans susceptible to injury by certain types of oxidative stress. Bilberry, also known to many as Dyeberry, Huckleberry, Trackleberry, Whortleberry or Wineberry grows naturally in many regions of the world including Canada, Europe and the United States. The ripe berries of the bilberry plant are most frequently used in the production of herbal extracts. The active constituents in bilberries are anthocyanosides, a flavonoid complex. Anthocyanosides are powerful antioxidants that support normal formation of connective tissue and strengthen capillaries in the body.

In one preferred embodiment of the present invention, the composition is comprised of the above mentioned herbal ingredients in the following approximate proportions relative to each other: about 0.01%-10% by volume Vitamin B3-6-12, Vitamin C, Vitamin E, CoQ10, Acai, Rosamarinic acid from Oregano, Peppermint, Lemon Balm, Lutien, Selenium and Bilberry extracts. However, it should be understood that the invention is not limited in this regard, and the relative amounts of these ingredients can be varied to achieve similar results.

After the herbal extracts are added 522 per the desired formula and mixed 524 thoroughly with the Guaraná liquid extract carrier the finished product may then be packaged 526, labeled and samples taken for quality assurance purposes.

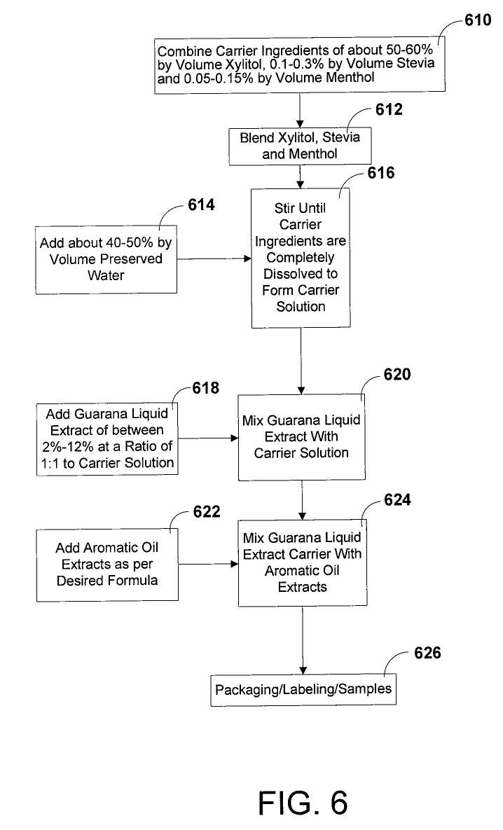

Further embodiments of the present invention, described with reference to FIG. 6, may comprise combining 610 carrier ingredients comprising from about 50-60% by volume Xylitol, 0.1-0.3% by volume Stevia and 0.05-0.15% by volume Menthol and blending 612 the Xylitol, Stevia and menthol thoroughly. In some embodiments, from about 40% to 50% by volume of “preserved water” which has been preheated to between 80-90 degrees Celsius may then be added 614 to the blended carrier ingredients and the heated preserved water and ingredients may then be stirred 616 until the ingredients are completely dissolved forming a carrier solution.

Once the carrier solution has been allowed to cool, a guaraná liquid extract of between 2% to 12% may then be added 618 to the carrier solution at a ratio of 1:1 and mixed 620 thoroughly. In some embodiments of the present invention an Aromatic Oil extract may then be added 622 to the guaraná carrier and mixed 624 thoroughly using methods known in the art.

Aromatic oils are fluid, colorless oils with a distinctly penetrating scent. Research has shown that the smell of aromatic oils can relax tension and relieve fatigue while increasing alertness and the ability to focus. Some embodiments of the present invention may combine guaraná and other herbal extracts with aromatic oils such as basil oil, bay oil, tea tree oil, sage oil, tea rose oil, tuberose oil, vetivert, winter green oil, ylang-ylang oil, sandal wood oil, spearmint oil, peppermint oil, tegetas oil, tangarin oil, termeric oil, aniseed oil and clove oil.

The finished product may then be packaged 626, labeled and samples taken for quality assurance purposes.

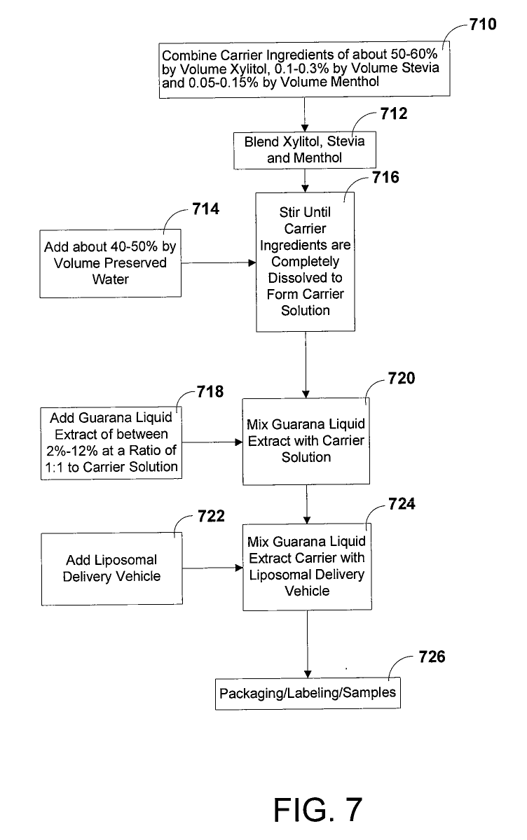

Further embodiments of the present invention, described with reference to FIG. 7, may comprise combining 710 carrier ingredients comprising from about 50-60% by volume Xylitol, 0.1-0.3% by volume Stevia and 0.05-0.15% by volume Menthol and blending 712 the Xylitol, Stevia and menthol thoroughly. In some embodiments, from about 40% to 50% by volume of “preserved water” which has been preheated to between 80-90 degrees Celsius may then be added 714 to the blended carrier ingredients and the heated preserved water and ingredients may then be stirred 716 until the ingredients are completely dissolved forming a carrier solution.

Once the carrier solution has been allowed to cool, a guaraná liquid extract of between 2% to 12% may then be added 718 to the carrier solution at a ratio of 1:1 and mixed 720 thoroughly. In some embodiments of the present invention a Liposomal Delivery Vehicle may then be added 722 to the guaraná carrier and mixed 724 thoroughly using methods known in the art.

The liposomal delivery system of the present invention may be a drug preparation that contains the active drug inside very tiny, fat-like particles. This form is easier for the body to absorb and allows more of a drug to get to the target area of the body. Liposomal drugs may have fewer side effects and work better than other forms of the drug.

Liposomes are used for drug delivery due to their unique properties. A liposome encapsulates a region on aqueous solution inside a hydrophobic membrane; dissolved hydrophilic solutes cannot readily pass through the lipids. Hydrophobic chemicals can be dissolved into the membrane, and in this way liposomes can carry both hydrophobic molecules and hydrophilic molecules. To deliver the molecules to sites of action, the lipid bilayer can fuse with other bilayers such as the cell membrane, thus delivering the liposome contents.

Liposomes can also be designed to deliver drugs in other ways. Liposomes that contain low (or high) pH can be constructed such that dissolved aqueous drugs will be charged in solution (i.e., the pH is outside the drug's pI range). As the pH naturally neutralizes within the liposome, the drug will also be neutralized, allowing it to freely pass through a membrane. These liposomes work to deliver a drug by diffusion rather than by direct cell fusion.

In accordance with another aspect of the present invention, the combination of herbal extracts can be further combined with liposomes, which may act as a carrier of the herbal composition. Preferably, the resulting combination is delivered through a spray or drop applied under the tongue. In some embodiments of the present invention, the drops may be delivered through an oral syringe. Among the advantages of using liposomes to deliver the composition are the resulting rapid absorption of the active ingredients, and the ease of use.

The formulations of the present invention may also be buffered. The buffer may include citrate or phosphate buffer. The formulation may further comprise a sweetener. Preferably, the sweetener is mannitol, saccharin, and/or saccharin sodium. The formulation may also further comprise a flavoring agent such as menthol.

The formulations of the present invention may further comprise a penetration enhancer such as chitosan. Preferably, the formulation is suitable for sublingual administration. The formulation may further comprise a mucoaguaranárant. The mucoaguaranárant may include, but is not limited to chitosan, polyvinyl pyrrolidone, and/or gelatin.

In another embodiment, the sublingual and buccal administration of guaraná may take the form of a paste or gel. The paste or gel may be applied under the tongue or buccally between the cheek and gum. The viscosity of the paste or gel can be adjusted to allow for better retention.

In some embodiments of the present invention, the guaraná composition may be formulated as lozenges or chewing gum. Such compositions may typically also include additional components such as a binder, a humectant, and flavoring agents such as sweeteners, artificial or natural fruit flavors, oils, and the like. Coloring may also be included.

In yet another embodiment, the sublingual administration of guaraná may comprise a device such as a sublingual patch. The patch may be placed under the tongue. The patch may have aguaranásive qualities to prevent the movement, loss or swallowing of the patch. The patch may be ingestible in case of accidental swallowing or to allow for easy disposal of the patch.

Further embodiments of the present invention may comprise tablets that disintegrate or dissolve rapidly in the consumer's mouth without the use of water and are convenient for consumers with swallowing difficulties, and in situations where water is not available. For these specially designed formulations, the small volume of saliva that is available is sufficient to disintegrate or dissolve a tablet in the oral cavity. The guaraná, or other herbal supplement released from these tablets may be absorbed partially or entirely into the systemic circulation from the buccal mucosa or sublingual cavity, or may be swallowed as a solution to be absorbed from the gastrointestinal tract.

One of the challenges of sublingual and buccal administration involves controlling the rate of release of the supplement. If the supplement is released more rapidly than it can be absorbed through the mouth, its concentration will increase in the saliva and it will be swallowed, whereupon it will be absorbed in the gut as though it were given in a conventional oral dosage form. Thus, it will be appreciated that control of the release rate will affect the supplement's bioavailability and variability in absorption between consumers.

A further aspect of the invention provides compositions and dosage forms especially adapted for buccal and sublingual administration of guaraná. Guaraná may be better absorbed in a more acidic environment than the neutral environment of the mouth. Acidifying the saliva, preferably to a pH between 2 and 7, may improve the absorption of guaraná.

Accordingly, preferred embodiments of the compositions and dosage forms of the present invention are able to acidify the local environment in the sublingual cavity or buccal cavity during the desired supplement release period. Such dosage forms contain an effective acidifying amount of an acidulant. An acidulant is an excipient that acidifies the local environment around the dosage form or composition after it has been put in the consumer's mouth. It need not acidify the saliva in all regions of the sublingual or buccal cavity to be effective, only the saliva that provides direct fluid communication between the surface of the dosage form from which the guaraná is released and adjacent oral mucosa. Acidulants are approved or generally recognized as safe excipients for use in oral supplement administration. Any approved or safe organic acid may be suitable, such as ascorbic acid, benzoic acid, citric acid, fumaric acid, lactic acid, malic acid, sorbic acid and tartaric acid.

In some embodiments of the present invention, it is contemplated that guaraná may be combined with inactive ingredients. Such ingredients may be necessary to add bulk to the guaraná preparation, to bind the preparation, to add color or flavor to the preparation or to prevent degradation or growth of contaminants.

In some embodiments, the composition may further comprise a pharmaceutically acceptable excipient. In certain embodiments, the pharmaceutically acceptable excipient is selected from the group consisting of: diluents, binders, glidants, lubricants, colorants, flavorants, coating materials, or combinations thereof.

It is also contemplated that some embodiments of the present invention may comprise other active ingredients in addition to guaraná, which may be added to the guaraná preparation of the present invention. The addition of any other active ingredient or ingredients is contemplated except where limited by the prior art. Such added active ingredients may augment the effectiveness of guaraná in alleviating or ameliorating headaches and pains. For example, it is contemplated that analgesics or anesthetics may be added to the guaraná preparation.

Several acceptable methods of sublingual administration are well known to those who are skilled in the art. The choice of method of sublingual administration method will be determined in part by the consumer. The following methods of administration are merely exemplary and in no way limit the present invention.

In one embodiment, the present invention provides a method of providing fast benefits from herbal supplements, including guaraná, comprising administering to a subject in need thereof an effective amount of supplement, by administering a drop onto the subject's sublingual mucosa.

In yet another aspect, the invention provides a method of providing fast benefits from herbal supplements, including guaraná, comprising administering to a subject in need thereof an effective amount of supplement, by spraying the supplement onto the subject's sublingual mucosa, i.e., the formulation may be sprayed directly onto the tissue under the consumer's tongue or drops may be delivered through an oral syringe directly onto the tissue under the consumer's tongue. By administering the guaraná directly to the sublingual mucosa, the consumer can experience fast and even immediate benefits, while still maintaining a high level of bioavailability.

In another embodiment, the invention provides a metered dose dispensing system for the administration of a guaraná liquid spray formulation, which may comprise guaraná extract, additional herbal extracts, buffered water and a polar organic solvent. The polar organic solvent may be present in an amount sufficient to enhance the solubility of the guaraná in the water. The metered dose dispensing system comprises a sealed container fitted with a metering pump, an actuator and a channeling device. Preferably, the metered dose dispensing system contains a metering chamber which is adapted for dispensation with the container in the upright orientation, and wherein the metering chamber is in communication with the formulation by means of a dip-tube.

Embodiments of the present invention, besides allowing to considerably reduce the therapeutic dose, present the additional advantage of improving the quickness of the beneficial effects.

The formulations according to the invention are prepared according to the known teachings and the methods generally employed in the field.

The terms and expressions which have been employed in the foregoing specification are used therein as terms of description and not of limitation, and there is no intention in the use of such terms and expressions of excluding equivalence of the features shown and described or portions thereof, it being recognized that the scope of the invention is defined and limited only by the claims which follow.

What is claimed and desired to be secured by Letters Patent is:

1. A therapeutic composition comprising:
a carrier; and a Guaraná extract.

2. The composition of claim 1, wherein the carrier comprises: Xylitol, Stevia, Menthol and Preserved Water.

3. The composition of claim 2, wherein the carrier comprises:
from about 50% to about 60% by carrier volume of Xylitol;
from about 0.1% to about 0.3% by carrier volume of Stevia;
from about 0.05% to about 0.09% by carrier volume of Menthol; and
from about 40% to about 50% by carrier volume of Preserved Water.

4. The composition of claim 2, wherein said Xylitol, said Stevia, said Menthol and said Preserved Water are mixed together to form a carrier.

5. The composition of claim 4, wherein said carrier is mixed with from about 2% to about 12% Guaraná liquid.

6. The composition of claim 5, wherein the carrier is mixed with the extract at a ratio of 1:1.

7. The composition of claim 1, wherein the Guaraná extract is a liquid extract.

8. The therapeutic composition according to claim 1, further comprising an additional supplement.

9. The therapeutic composition according to claim 1, further comprising an additional supplement wherein said additional supplement is selected from the group consisting of: Vitamin B12, Vitamin C, Vitamin E, Folic Acid, Capsaicin, Ginseng, CoQ10, Magnesium, Zinc, Potassium, Niacin, Thiamin, Avena Sativa or combinations thereof.

10. The therapeutic composition according to claim 1, further comprising an additional supplement wherein said additional supplement is selected from the group consisting of: Vitamin B3, Vitamin B6, Vitamin B12, Hawthorn, Ginseng, Cayenne, Bilberry, Garlic, Evening Primrose Oil (EPO), Coleus Forskohlii, Sage or combinations thereof.

11. The therapeutic composition according to claim 1, further comprising an additional supplement wherein said additional supplement is selected from the group consisting of: Ginko Biloba, Gotu Kola, CoQ10, Vitamin E, Vitamin C, Folate, Ginseng, Lemon Balm, Theobroma Cacao or combinations thereof.

12. The therapeutic composition according to claim 1, further comprising an additional supplement wherein said additional supplement is selected from the group consisting of: White Willow Bark, Feverfew, Lavender, Cat's Claw, Vitamin C, Gingko, Ginseng, Rosemary Puree, Milk Thistle, a generic analgesic or combinations thereof.

13. The therapeutic composition according to claim 1, further comprising an additional supplement wherein said additional supplement is selected from the group consisting of: Vitamin B, Ginko, Horny Goat Weed, Cnidium, Cistanche Bark, Vitamin A & E, Selenium, Zinc, Magnesium, Maca, Damiana or combinations thereof.

14. The therapeutic composition according to claim 1, further comprising an additional supplement wherein said additional supplement is selected from the group consisting of: Capsaicin, Ginseng, Hoodia Gordonii, Vitamin B12, Vitamin C, Vitamin E, Garcinia Cambogia, Cereus Grandiflorus or combinations thereof.

15. The therapeutic composition according to claim 1, further comprising an additional supplement wherein said additional supplement is selected from the group consisting of: Lithium, Skullcap, Valerian, Passion Flower, Polygala, Vitamin E, Hawthorne, Ginkgo, Cyracos, Passion Flower, Kava Kava or combinations thereof.

16. The therapeutic composition according to claim 1, further comprising an additional supplement wherein said additional supplement is selected from the group consisting of: MSM, Selenium, Glucosamine, HCL, Yucca, Chondroitin, Vitamin E, Boswellia Serrata, White Willow, Feverfew, Devil's Claw, Turmeric or combinations thereof.

17. The therapeutic composition according to claim 1, further comprising an additional supplement wherein said additional supplement is selected from the group consisting of: Golden Seal, Nettle, Ginkgo, Vitamin C, Barberry, Echinacea, Eucalyptus, Linden, Perilla, Butterbur or combinations thereof.

18. The therapeutic composition according to claim 1, further comprising an additional supplement wherein said additional supplement is selected from the group consisting of: Vitamin B3-6-12, Vitamin C, Vitamin E, CoQ10, Acai, Rosamarinic acid from Oregano, Peppermint, Lemon Balm, Lutien, Selenium, Bilberry or combinations thereof.

19. The therapeutic composition according to claims 1-12, further comprising an aromatic oil wherein said aromatic oil is selected from the group consisting of: basil oil, bay oil, tea tree oil, sage oil, tea rose oil, tuberose oil, vetivert, winter green oil, ylang-ylang oil, sandal wood oil, spearmint oil, peppermint oil, tegetas oil, tangarin oil, termeric oil, aniseed oil, clove oil or combinations thereof.

20. The therapeutic composition according to claims 1, further comprising liposomes for liposomal administration of said composition.

21. The therapeutic composition according to claims 1, in the form of a drop, suitable for sublingual administration.

22. The therapeutic composition according to claims 1, in the form of a spray, suitable for sublingual administration.

23. The therapeutic composition according to claims 1, in a form selected from the group consisting of: a tablet, an oral fast dissolving tablet, a lozenge, chewing gum, paste and gel, suitable for sublingual or buccal administration.

24. A method of preparing a therapeutic composition, comprising the steps of:
combining from about 50% to about 60% by carrier volume of Xylitol; from about 0.1% to about 0.3% by carrier volume of Stevia; from about 0.05% to about 0.09% by carrier volume of Menthol;
blending said Xylitol, said Stevia and said Menthol;
adding from about 40% to about 50% by carrier volume of preserved water heated to between 80-90 degrees Celsius;
stirring said Xylitol, said Stevia and said Menthol into said preserved water until said Xylitol, said Stevia and said Menthol are completely dissolved into said preserved water and allowed to cool forming a carrier;
adding from about 2% to about 12% Guaraná liquid extract to said carrier at a ratio of 1:1 to said carrier; and
mixing said from about 2% to about 12% Guaraná liquid extract and said carrier thoroughly to form said therapeutic composition.

25. The method according to claim 18, further comprising adding additional supplements to said therapeutic composition.

26. A method of extracting Guaraná extract for human consumption from the Paulinia Cupana plant comprising the steps of:
Grinding said Paulinia Cupana plant;
Extracting said Guaraná from said Paulinia Cupana plant;
Filtering said extracted Guaraná;
desolventizing said extracted Guaraná;
blending said extracted Guaraná with glycerol and ethanol; and
filtering said extracted Guaraná.
